(19) United States
(12) Patent Application Publication
(10) Pub. No.: US 2025/0205034 A1
(43) Pub. Date: Jun. 26, 2025

(54) SURGICAL APPLICATION SYSTEM FOR A NECK LIFT AND METHOD OF INSERTION SAME

(71) Applicant: SHAARE ZEDEK SCIENTIFIC LTD., Jerusalem (IL)

(72) Inventors: Yoav GRONOVICH, Maale Hahamisha (IL); Yehuda BACHAR, Yehud-Monosson (IL)

(21) Appl. No.: 19/078,484

(22) Filed: Mar. 13, 2025

Related U.S. Application Data
(63) Continuation-in-part of application No. PCT/IL2023/050997, filed on Sep. 13, 2023.
(60) Provisional application No. 63/405,935, filed on Sep. 13, 2022.

(51) Int. Cl. A61F 2/00; A61B 17/00

(52) U.S. Cl. CPC A61F 2/0063; A61B 2017/00792; A61F 2002/0072; A61F 2230/0023

(57) ABSTRACT

A surgical application system for neck lift surgery is disclosed. The surgical application system comprises: an applicator, insertable into a submental triangle of a patient undergoing neck lift surgery; and a neck lift sling, detachably connected to a frame of the applicator, wherein the neck lift sling is spread on and supported by the applicator frame, wherein the applicator frame may be shaped to fit the submental triangle of the patient.

### CROSS-REFERENCE TO RELATED APPLICATIONS

This application is a Continuation in Part of PCT Patent Application No. PCT/IL2023/050997 filed on Sep. 13, 2023, which claims the benefit of priority of U.S. Provisional Patent Application No. 63/405,935, titled “SURGICAL APPLICATION SYSTEM FOR A NECK LIFT AND METHOD OF INSERTION SAME”, filed Sep. 13, 2022, the contents of which are incorporated herein by reference in their entirety.

### FIELD OF THE INVENTION

The invention relates to the field of neck lift surgery. More specifically, the present invention relates to surgical application systems for a neck lift and methods of insertion thereof.

### BACKGROUND OF THE INVENTION

In plastic surgery, a neck lift, or lower rhytidectomy, is a surgical procedure that improves visible signs of aging in the jawline and neck. A neck lift may remove excess fat and skin in the lower face that creates jowls, remove fatty deposits under the chin, tighten loose neck skin, adjust muscle banding in the neck, for example that create abnormal contours, and/or the like. Factors that cause the need for a neck lift may include heredity, gravity, environmental conditions, stress, and/or the like. These factors may cause excess wrinkling of the neck skin, such as referred to colloquially as “turkey wattle” or a double chin, and impair jowl lines.

Other procedures related to lower rhytidectomy are cervicoplasty and platysmaplasty, which are correction of submental ptosis sometimes necessary to reverse the effects of aging. Cervicoplasty and platysmaplasty may be performed together with facial rhytidectomy, when surgeons may need to address the cervical region.

Originating from the pectoralis major muscle fascia, the platysma is a layer of muscle that has multiple insertions. Moving anterior to posterior, the muscle is anchored to the mentum and the inferior mandibular border and meets the orbicularis oris laterally and then the depressor anguli oris. Platysmal meshing with the depressor anguli oris contributes to the superficial muscular aponeurotic system (SMAS), highlighting its importance when attempting to reverse facial aging.

A neck lift may be performed by a horizontal submental incision for lipectomy, with excision or plication of hypertrophic anterior platysmal bands, and wide subcutaneous cervical dissection. This may correct fasciocutaneous ligaments extending from the dermis to the facial fascia, and osseocutaneous ligaments extend from the dermis to the periosteum.

Adipose tissue may comprise subcutaneous and subplatysmal fat tissue. Increased amounts of subcutaneous fat may be observed with weight gain, aging, lipodystrophies, and the like. Subplatysmal fat is far more vascular and fibrous than subcutaneous fat and is visualized after incising the platysma muscle. This difference may result in reduced efficacy of liposuction of subplatysmal fat.

Accordingly, there is a need for a simple and accurate way for the insertion of a neck lift sling, that will ensure satisfactory results of the neck lift surgery.

### SUMMARY OF THE INVENTION

Some aspects of the invention may be directed to a surgical application system for neck lift surgery, comprising: an applicator, insertable into a submental triangle of a patient undergoing neck lift surgery; and a neck lift sling, detachably connected to a frame of the applicator, wherein the neck lift sling is spread on and supported by the applicator frame, wherein the applicator frame may be shaped to fit the submental triangle of the patient.

In some embodiments, the surgical application system further comprises a delivery tube shaped to encompass the applicator and the neck lift sling, open from at least one end. In some embodiments, the delivery tube's crosse section is sized to compress the applicator. In some embodiments, the delivery tube is made from an elastic material.

In some embodiments, the applicator frame has two lateral extensions each having a lateral edge. In some embodiments, the applicator frame is open at one lateral edge and the applicator further includes a lock configured to lock the open edge and prevent the movement of the neck lift sling during insertion of the system, and to be removed and allow a detachment of the neck lift sling from the applicator frame during extraction of the frame from the submental triangle.

In some embodiments, the applicator frame is made from a flexible plastic material. In some embodiments, the applicator comprises at least one of: a medical grade polymer and a medical grade alloy.

In some embodiments, the neck lift sling is a perforated sheet having a shape fitting the shape of the applicator frame. In some embodiments, the neck lift sling is a perforated sheet having a body and two lateral sling extensions, each coupled to a respective side edge of two sides of the body each projecting away from the center of the sling body in a posterior lateral orientation of between 45 and 90 degrees from the anterior-posterior axis, and curved slightly towards the narrow anterior edge to fit the submandibular triangle and extend to near each ear of the patient.

In some embodiments, the neck lift sling is threaded at its circumferential edge on the applicator frame. In some embodiments, at least portions of the circumferential edge of the neck lift sling are folded to form loops and wherein the frame is inserted inside the loops. In some embodiments, at least portions of the circumferential edge of the neck lift sling are glued to the applicator frame using a glue capable of being unglued by a slight pulling action.

In some embodiments, the neck lift sling comprises at least one biocompatible material. In some embodiments, the neck lift sling comprises at least one bio-absorbable material. In some embodiments, the neck lift sling comprises a net or a fabric having gaps size of between 0.1 mm to 10 mm. In some embodiments, the fabric is a woven fabric or nonwoven fabric. In some embodiments, the fabric or net includes elastic fibers and plastic fibers.

Some additional aspects may be directed to a medical method for inserting a sling in neck lift surgery, comprising: making incisions near each ear of a patient along a posterior edge of the tragus down through the lobule and posteriorly at the retroauricular sulcus through the hairline, and near a chin of the patient parallel to the mental crease line and five millimeters posterior to the crease line, wherein the incision cut near the chin has a length of 4 centimeters; creating a subcutaneous pocket between said incisions; inserting surgical application system, according to any one of the embodiments disclosed herein, to said subcutaneous pocket; slightly pulling the applicator frame from said subcutaneous pocket, from one of the incisions, while detaching said sling from said frame; and anchoring the ends of two extensions of the sling near the subcutaneous connective tissue near the ear, and a front edge of the sling to the subcutaneous tissue near the chin.

Some additional aspects may be directed to a medical method for inserting a sling in neck lift surgery, comprising: making incisions near each ear of a patient along a posterior edge of the tragus down through the lobule and posteriorly at the retroauricular sulcus through the hairline, and near a chin of the patient parallel to the mental crease line and five millimeters posterior to the crease line, wherein the incision cut near the chin has a length of 4 centimeters; creating a subcutaneous pocket between said incisions; inserting surgical application system, according to any one of the embodiments disclosed herein, to said subcutaneous pocket; slightly pulling the delivery tube from said subcutaneous pocket, from one of the incisions, leaving the applicator and the sling inside said subcutaneous pocket; slightly pulling the applicator frame from said subcutaneous pocket, from one of the incisions, while detaching said sling from said frame; and anchoring the ends of two extensions of the sling near the subcutaneous connective tissue near the ear, and a front edge of the sling to the subcutaneous tissue near the chin.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A, 1B, 1C, 1D and 1E are illustrations of a surgical application system for a neck lift, an applicator, a sling, and an insertion tube, according to some embodiments of the invention;

FIGS. 2A, 2B, 2C and 2D are illustrations of various optional geometries for the applicator and the sling, according to some embodiments of the invention;

FIG. 3 is a flowchart of a medical method for inserting a sling in neck lift surgery, according to some embodiments of the invention;

FIGS. 4A and 4B are illustrations of two views of incisions for neck lift sling surgery, according to some embodiments of the invention; FIG. 4C is an illustration of a step of pulling the insertion tube in the method of FIG. 3 according to some embodiments of the invention; and

FIGS. 5A, 5B, and 5C are illustrations of some of the steps of the method of FIG. 3 according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

Some aspects of the invention may be directed to a surgical application system for neck lift surgery and a method of inserting the surgical application system into the submental triangle of a patient undergoing the neck lift surgery. The system may include two separatable components, an applicator, and a neck lift sling, detachably connected to a frame of the applicator. The system as a whole is inserted into a subcutaneous pocket made in the submental triangle between two incisions near each ear of the patient. In some embodiments, the applicator may allow simple and accurate insertion of the neck lift sling, to the required location in the subcutaneous pocket. After the insertion to the correct placement of the system, the neck lift sling may be detached from the frame of the applicator and the applicator may be pulled out of the subcutaneous pocket, via one of the incisions. The outcome of the process is the correct placement of a spread neck lift sling in the subcutaneous pocket. The surgery may be ended when the spread neck lift sling is anchored, as discussed herein below and the incisions are stitched.

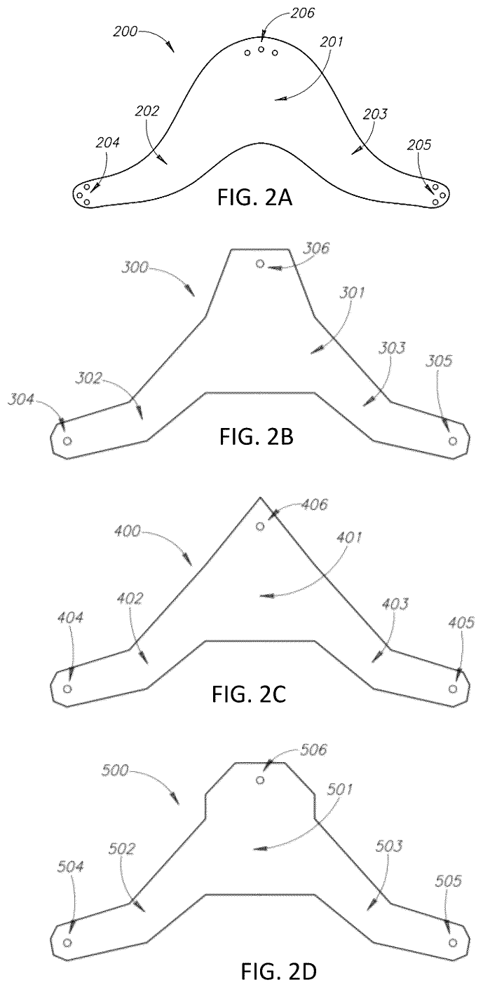

Reference is now made 1A, 1B, 1C, 1D, and 1E which are illustrations, according to some embodiments, of a surgical application system for a neck lift, an applicator, a sling, and an insertion tube, respectively. A surgical application system 10 for neck lift surgery may include an applicator 50, insertable into a submental triangle of a patient undergoing neck lift surgery, and a neck lift sling 100, detachably connected to a frame 51 of applicator 50, wherein the neck lift sling 100 is spread on and supported by applicator frame 51. In some embodiments, applicator frame 51 may be shaped to fit the submental triangle of the patient, as illustrated. Some additional nonlimiting examples for various shapes of application frames and neck lift slings are given in FIGS. 2A, 2B, 2C, and 2D.

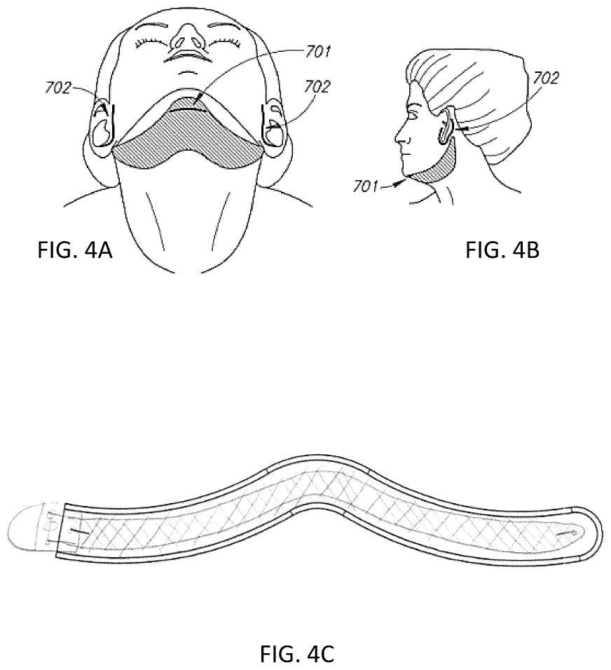

In some embodiments, applicator frame 51 has two lateral extensions 52 and 53 each having a corresponding lateral edge 54 and 55. In some embodiments, applicator frame 51 is open at one lateral edge 54 and applicator 50 further includes a lock 58 configured to lock open edge 54 and prevent the movement of neck lift sling 100 during insertion of system 10, and to be removed and allow a detachment of neck lift sling 100 from applicator frame 51 during extraction of the frame from the submental triangle. In some embodiments, lock 58 may be or may include any element or device that may allow closing and opening of open lateral edge 54. In a non-limiting example, lock 58 may include a body 59 for holding the open ends of farm 51 and a cup 61, as illustrated. In other nonlimiting examples, lock 58. In some embodiments, lock 58 may be connected to neck lift sling 100 via thread 25, as discussed with respect to FIGS. 4A-4C.

In some embodiments, neck lift sling 100 may be secured to applicator frame 51 using a loop created lateral edge 55. In this configuration, at lateral edge 54 (e.g., the open edge) of application frame 51, frame 51 may not be connected to at least a portion of neck lift sling 100, forming a free edge of neck lift sling 100. In some embodiments, the free edge of neck lift sling 100 may be connected to cup 61.

In some embodiments, applicator frame 51 is made from a flexible material adapted to fit the submental triangle of the patient. For example, applicator frame 51 and/or lock 58 of applicator 50 may be made or may include at least one of: a medical grade polymer and a medical grade alloy. In some embodiments, applicator frame 51 and/or lock 58 may be coated with a medical grade polymer. For example, the medical grade metal may include titanium (Ti), stainless steel (SS), Nickel titanium (nitinol) alloy, alloys thereof, metal alloys, and/or the like. In another example, the medical grade polymer may be medical grades of polyvinyl chloride, polyethylene, polyether ether ketone, polycarbonate, polyetherimide, polysulfone, polypropylene, polyurethane, and/or the like.

In some embodiments, neck lift sling 100 is a perforated sheet comprising a net or a fabric having gaps size/pore size of at least 0.1 mm, for example, between 0.1 mm to 10 mm (or any value in between), between fibers of the net or fabric. For example, the gaps/pore size is of between 0.1 mm to 1 mm, between 0.5 mm to 2 mm, 1 mm to 3 mm, 0.3 mm to 2.5 mm or any value in between the ranges.

In some embodiments, the fabric may be a woven or nonwoven fabric. In some embodiments, the fabric or net may include two different types of fibers, for example, elastic fibers and plastic fibers. In some embodiments, the elastic fibers are configured to provide elasticity allowing the sling to fit to the substations pocket and the plastic fibers may provide the support for the neck lift.

In some embodiments, neck lift sling 100 comprises at least one biocompatible material. For example, biocompatible material may be any medical grade polymer, such as, medical grades of polyvinyl chloride, polyethylene, polyether ether ketone, polycarbonate, polyetherimide, polysulfone, polypropylene, polyurethane, and/or the like.

In some embodiments, the neck lift sling comprises at least one bio-absorbable material. Specific bio-absorbable polymers may be absorbable polyglactin 910, polydioxanone, polyglycolic-acid, polylactic acid, poly (lactic-co-glycolic acid), polycaprolactone, polyglycolide, polydioxanone, polyhydroxybutyrate, and/or the like.

In some embodiments, neck lift sling 100 is threaded at its circumferential edge 108 on applicator frame 51, as illustrated. For example, perforations 109 at the circumferential edge of neck lift sling 100 may be threaded on applicator frame 51. In some embodiments, at least portions of circumferential edge 108 of neck lift sling 100 are folded to form loops (not illustrated) and frame 51 is inserted inside the loops. In some embodiments, at least portions of circumferential edge 108 of neck lift sling 100 are glued to applicator frame 51 (not illustrated) using a glue capable of being unglued by a slight pulling action.

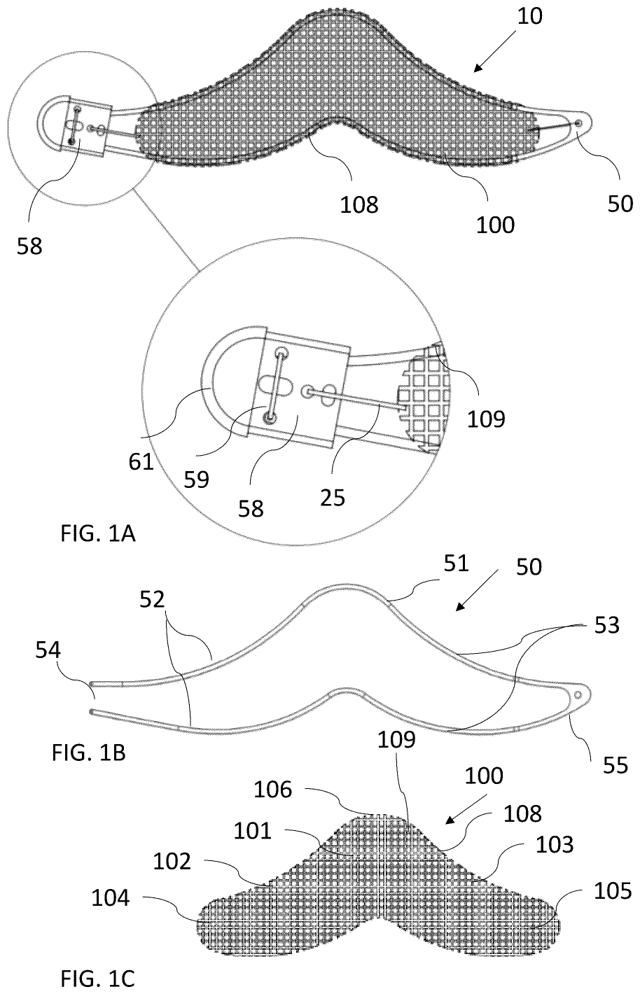

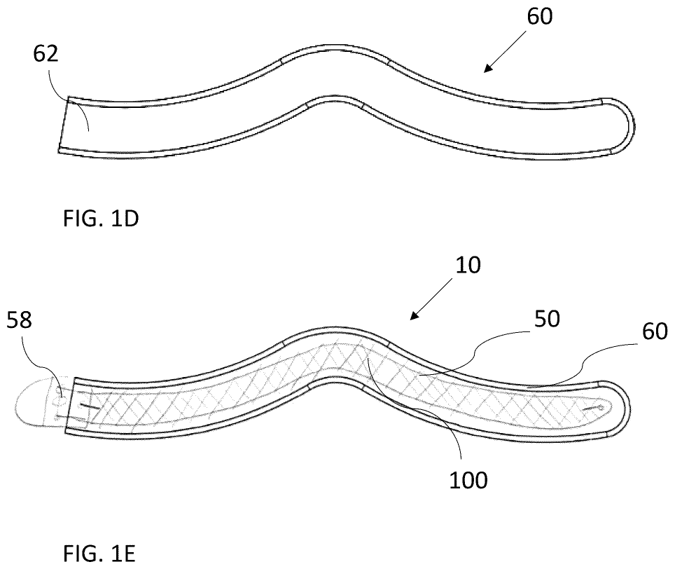

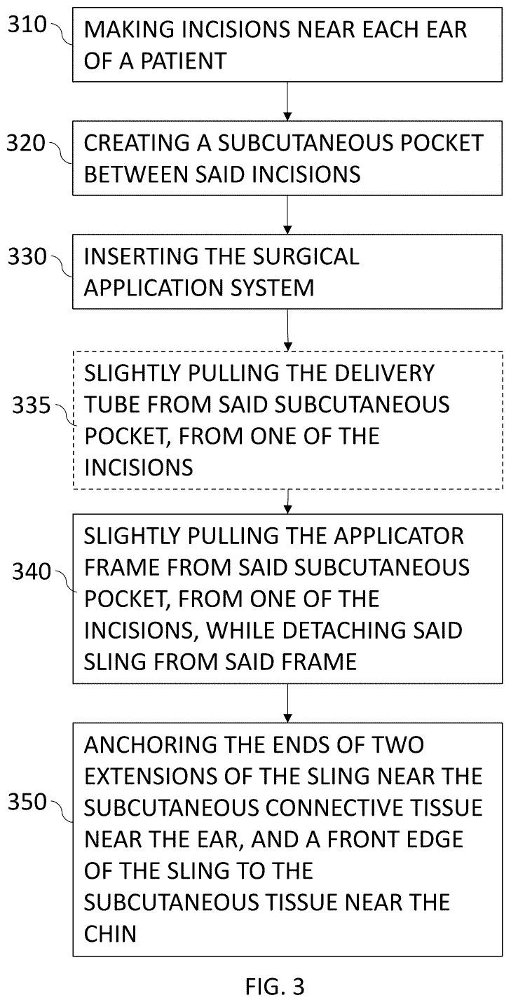

In some embodiments, neck lift sling 100 is a perforated sheet having a shape fitting the shape of the applicator frame. In some embodiments, neck lift sling 100 is a perforated sheet having a body 101 and two lateral sling extensions 102 and 103, as illustrated, each coupled to a respective side edge 104 and 105 of two sides of body 101 each projecting away from the center of sling body 101 in a posterior lateral orientation of between 45 and 90 degrees from the anterior-posterior axis, and curved slightly towards the narrow anterior edge to fit the submandibular triangle and extend to near each ear of the patient. In some embodiments, slings body 101 may further include a front edge 106 to be placed in subcutaneous tissue near the chin. In some embodiments, surgical application system 10 may further include a delivery tube 60 shaped to encompass the applicator and the neck lift sling, open from at least one end 62, as illustrated in FIGS. 1D and 1E. Tube 60 may allow an easier insertion of applicator 50 into the submental triangle of the patient undergoing neck lift surgery, as discussed herein below with respect to the method of FIG. 3 and illustrated in FIG. 4C. In some embodiments, delivery tube's 60 crosse section is sized to compress the applicator, as to further ease the insertion of system into a pocket in the submental triangle. Tube 60 may be a substantially flat elastic tube, made form an elastic material, for example, silicone, polyurethane, etc. In some embodiments, delivery tube 60 may further include a metallic wire to be inserted inside the tube (e.g., casted with, glued, threaded, etc.) in order to prevent the twisting of delivery tube 60.

In some embodiments, holes (illustrated in FIGS. 2A-2D) are prefabricated in the corners of sling 100 for anchoring to the chin and sub-aural connective tissue. For example, three holes are prefabricated in each lateral and medial end of the extensions of sling 100, and at the anterior corner. Each hole may be of 2 millimeters diameter and located 3 millimeters from the edge of sling 100. In some embodiments, the holes may be between 0.5 and 5 millimeters in diameter and located between 0.2 and 20 millimeters from an edge and/or corner of the sling. The number of holes, their location, and their size may be chosen according to ease of attachment, to the particular anatomy of a subject, for a particular method of attachment, for a particular configuration of components, for a particular shape of sling, and/or the like.

In some embodiments, holes are custom cut and/or punched in sling 100 for each patient. For example, sling 100 has long extension portions that are cut to fit the patient anatomy and holes are punched in near the cut ends to anchor the submandibular sling body of sling 100.

As should be understood by one skilled in the art, the shape of neck lift sling 100 and frame 51 are given as nonlimiting examples, and other shapes and geometries may be applied to neck lift sling 100 and frame 51. Some additional nonlimiting geometries for neck lift sling 100 and frame 51 are illustrated in FIGS. 2A-2D. In some embodiments, sling 100 and/or frame 51 is prefabricated in multiple sizes to fit different patients. For example, the TABLE 1 lists possible prefabricated sizes in centimeters.

TABLE 1

| Size Name | Anterior-Posterior Size | Lateral Size |
|---|---|---|
| XS | 4 | 14 |
| S | 5 | 17 |
| M | 6 | 20 |
| L | 7 | 25 |
| XL | 8 | 30 |
| XXL | 9 | 35 |

Reference is now made to FIGS. 2A-2D which are illustrations of nonlimiting examples for various geometries for the lift sling and the frame. In some embodiments, neck lift slings 200, 300, 400 and 500 are perforated sheets having a shape fitting applicator frame. In some embodiments, slings 200, 300, 400 and 500 are perforated sheets having corresponding bodies 201, 301, 401 and 501 and two lateral corresponding sling extensions 202/203, 302,303, 402/403 and 502/503, as illustrated, each coupled to a respective side edge 204/205, 304/305, 404/405 and 5054/505 of two sides of bodies 201, 301, 401 and 501 each projecting away from the center of sling bodies 201, 301, 401 and 501 in a posterior lateral orientation of between 45 and 90 degrees from the anterior-posterior axis, and curved slightly towards the narrow anterior edge to fit the submandibular triangle and extend to near each ear of the patient. In some embodiments, slings bodies 201, 301, 401 and 501 may further include a front edge 206, 306, 406 and 506 to be placed in subcutaneous tissue near the chin.

In some embodiments, each one of neck lift slings 200, 300, 400 and 500 may have a matching applicator holder 50. Alternatively, farm 51 of applicator 50 may be flexible enough to suit any one of neck lift slings 100, 200, 300, 400 and 500.

In some embodiments, neck lift slings 200, 300, 400 and 500 may include holes as discussed herein above. In some embodiments, neck lift slings 200, 300, 400 and 500 may be made from the same material as neck lift sling 100.

Reference is now made to FIG. 3 which is a flowchart of a method for inserting a sling in neck lift surgery according to some embodiments of the invention.

In step 310, incisions may be made near each ear of a patient along a posterior edge of the tragus down through the lobule and posteriorly at the retroauricular sulcus through the hairline, and near a chin of the patient parallel to the mental crease line and five millimeters posterior to the crease line, wherein the incision cut near the chin has a length of 4 centimeters, as illustrated in FIGS. 4A and 4B. In some embodiments, prior to making the incisions, the patient may be anesthetized and positioned supine on a surgical table. For example, the procedure may be done under local anesthesia, sedation or general anesthesia.

Reference is now made to FIGS. 4A and 4B, which are schematic illustration of views of incisions for neck lift sling surgery, according to some embodiments of the invention. The posterior edge of the tragus 702 may be marked down through the lobule and posteriorly at the retroauricular sulcus through the hairline. Mental crease line 701 may be marked five millimeters posterior to the crease, for a length of 4 centimeters. In some embodiments, the markings may be located at various locations and of various sizes according to ease of attachment, to the particular anatomy of a subject, for a particular method of attachment, for a particular configuration of components, for a particular shape of sling, and/or the like. For example, the markings may be located between 1 and 25 millimeters posterior to the mental crease for a length of between 1 and 10 centimeters.

In some embodiments, the incisions may be cut in the mental crease, then on the distal half of the retroauricular sulcus. In cases of excessive skin redundancy, the incision may be cut preauricularly to the lobule and the edge of the tragus.

In step 320, a subcutaneous pocket may be created, by local infiltration and undermining is performed. Local infiltration to the subcutaneous plane may be with NaCl 0.9% 100 cc, epinephrine 1:300,000, and local anesthetics when the procedure is done under local anesthesia. Undermining of the skin may be performed using Gorney scissors in a subcutaneous plane. Most of the dissection may be accomplished by spreading maneuvers of the scissors. The undermining may be performed from the three mentioned incisions: one mental and two retroauricular. In the mental incision, dissection may be performed also anteriorly to the chin for correction of ptosis and creation of space for the edge of slings 100-500.

In some embodiments, after the subcutaneous plane is created, meticulous hemostasis is done under direct vision. Pockets are packed with soaked gauzes, one from each incision, with povidine iodine and surgical application system 10 packaging is opened and system 10 is prepared for insertion.

In step 330, surgical application system 10 is inserted to said subcutaneous pocket. In some embodiments, gauzes are removed and the subcutaneous pocket is exposed from the mental incision using a retractor. In some embodiments, surgical application system 10 is inserted such that lateral edges 54 and 55 of applicator 50 are exposed via incisions 702 near each ear of a patient. After the insertion slings 100-500 are still spread on and supported by applicator frame 50 inside the subcutaneous pocket.

In some embodiments, when system 10 comprises delivery tube 60, the delivery tube may be slightly pulled from said subcutaneous pocket, from one of the incisions, leaving the applicator and the sling inside said subcutaneous pocket, in optional step 335. Delivery tube 60 may compress application 50 and sling 100 to ease the insertion of system 100. After the pulling of delivery tube 60, as illustrated in FIG. 4C, application 50 and sling 100 are left inside the pocket.

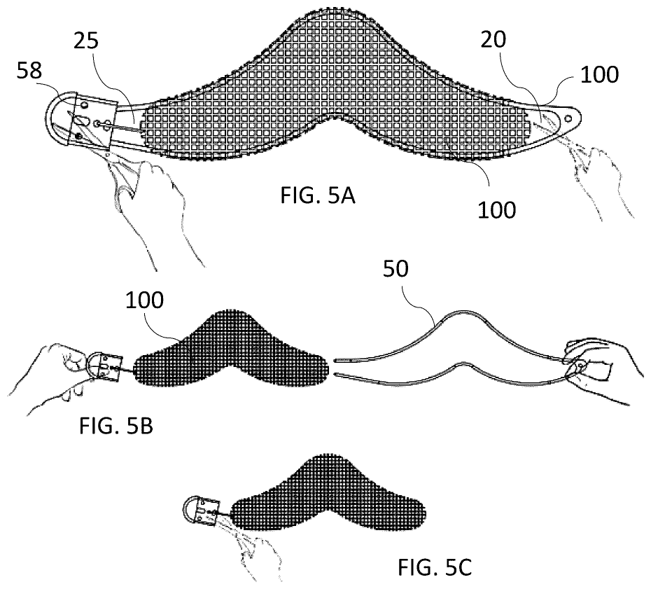

In step 340, farm 51 may be detached from slings 100-500 and then slightly be pulled from the subcutaneous pocket, as illustrated in FIGS. 5Am 5B and 5C. Trade 20 anchoring slings 100-500 to frame 50 may be cut via incision 702. Frame 51 may slightly be pulled from one incision 702 while lock 58 is being held, as illustrated in FIGS. 5A and 5B. In some embodiments, tread 25 anchoring slings 100-500 to lock 58 is cut via the other incision 702, as illustrated in FIG. 5C. In some embodiments, the outcome is a neck lift sling which is easily and precisely located at the subcutaneous pocket.

In step 350, the ends of two extensions of the sling may be anchored near the subcutaneous connective tissue near the ear, and a front edge of the sling to the subcutaneous tissue near the chin. In some embodiments, ends 104-504 and 105-505 may be anchored near the subcutaneous connective tissue near the ear near incision 702 and front edges 106-506 of the sling to the subcutaneous tissue near the chin at incision 701.

In some embodiments, the method may further include closing the incisions, for example, in two layers, and drains inserted from the retroauricular incisions for the first 24 hours. Dressings may be performed with an antibiotic ointment and gauzes, and a soft collar bandage may be worn. Antibiotics may be prescribed for the first 7 days, such as 1-gram cephalexin three times per day. Local treatment may be performed with an antibiotic ointment. Patient may be allowed to shower after the first 24 hours, and may be abstain from physical exercise on the first 4 weeks. Sutures are removed on postoperative day 7. The soft collar bandage.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A surgical application system for neck lift surgery, comprising:
an applicator, insertable into a submental triangle of a patient undergoing neck lift surgery; and
a neck lift sling, detachably connectable to a frame of the applicator, wherein, when connected to the frame of the applicator, the neck lift sling is spread on and supported by the applicator frame,
wherein the applicator frame is shaped to fit the submental triangle of the patient.

2. The surgical application system of claim 1, further comprising a delivery tube shaped to encompass the applicator and the neck lift sling, wherein the delivery tube is open from at least one end.

3. The surgical application system of claim 2, wherein the delivery tube's crosse section is sized to compress the applicator.

4. The surgical application system of claim 2, wherein the delivery tube is made from an elastic material.

5. The surgical application system of claim 1, wherein the applicator frame has two lateral extensions each having a lateral edge.

6. The surgical application system of claim 5, wherein the applicator frame is open at one lateral edge and the applicator further includes a lock configured to lock the open edge and prevent the movement of the neck lift sling during insertion of the system, and to be removed and allow a detachment of the neck lift sling from the applicator frame during extraction of the frame from the submental triangle.

7. The surgical application system according claim 1, wherein the applicator frame is made from a flexible plastic material.

8. The surgical application system of claim 7, wherein the applicator comprises at least one of: a medical grade polymer and a medical grade alloy.

9. The surgical application system of claim 1, wherein the neck lift sling is a perforated sheet having a shape fitting the shape of the applicator frame.

10. The surgical application system of claim 1, wherein the neck lift sling is a perforated sheet having a body and two lateral sling extensions, each coupled to a respective side edge of two sides of the body each projecting away from the center of the sling body in a posterior lateral orientation of between 45 and 90 degrees from the anterior-posterior axis, and curved slightly towards the narrow anterior edge to fit the submandibular triangle and extend to near each ear of the patient.

11. The surgical application system of claim 1, wherein the neck lift sling is threaded at its circumferential edge on the applicator frame.

12. The surgical application system of claim 1, wherein at least portions of the circumferential edge of the neck lift sling are folded to form loops and wherein the frame is inserted inside the loops.

13. The surgical application system of claim 1, wherein at least portions of the circumferential edge of the neck lift sling are glued to the applicator frame using a glue capable of being unglued by a slight pulling action.

14. The surgical application system of claim 1, wherein the neck lift sling comprises at least one biocompatible material.

15. The surgical application system of claim 1, wherein the neck lift sling comprises at least one bio-absorbable material.

16. The surgical application system of claim 1, wherein the neck lift sling comprises a net or a fabric having gaps size of between 0.1 mm to 10 mm.

17. The surgical application system of claim 16, wherein the fabric is a woven fabric or nonwoven fabric.

18. The surgical application system of claim 16, wherein the fabric or net includes elastic fibers and plastic fibers.

19. A medical method for inserting a sling in neck lift surgery, comprising:
(a) making incisions near each ear of a patient along a posterior edge of the tragus down through the lobule and posteriorly at the retroauricular sulcus through the hairline, and near a chin of the patient parallel to the mental crease line and five millimeters posterior to the crease line, wherein the incision cut near the chin has a length of 4 centimeters;
(b) creating a subcutaneous pocket between said incisions;
inserting surgical application system to said subcutaneous pocket, wherein the surgical application system comprises:
an applicator, insertable into a submental triangle of the patient;
a neck lift sling, detachably connectable to a frame of the applicator, wherein, when connected to the frame of the applicator, the neck lift sling is spread on and supported by the applicator frame;
a delivery tube shaped to encompass the applicator and the neck lift sling, wherein the delivery tube is open from at least one end, wherein the applicator frame is shaped to fit the submental triangle of the patient;
(c) slightly pulling the applicator frame from said subcutaneous pocket, from one of the incisions, while detaching said sling from said frame; and
(d) anchoring the ends of two extensions of the sling near the subcutaneous connective tissue near the ear, and a front edge of the sling to the subcutaneous tissue near the chin.

20. A medical method for inserting a sling in neck lift surgery, comprising:
making incisions near each ear of a patient along a posterior edge of the tragus down through the lobule and posteriorly at the retroauricular sulcus through the hairline, and near a chin of the patient parallel to the mental crease line and five millimeters posterior to the crease line, wherein the incision cut near the chin has a length of 4 centimeters;
creating a subcutaneous pocket between said incisions;
inserting surgical application system to said subcutaneous pocket,
wherein the surgical application system comprises:
an applicator, insertable into a submental triangle of the patient; and
a neck lift sling, detachably connectable to a frame of the applicator, wherein, when connected to the frame of the applicator, the neck lift sling is spread on and supported by the applicator frame,
and wherein the applicator frame is shaped to fit the submental triangle of the patient;
slightly pulling the delivery tube from said subcutaneous pocket, from one of the incisions, leaving the applicator and the sling inside said subcutaneous pocket;
slightly pulling the applicator frame from said subcutaneous pocket, from one of the incisions, while detaching said sling from said frame; and
anchoring the ends of two extensions of the sling near the subcutaneous connective tissue near the ear, and a front edge of the sling to the subcutaneous tissue near the chin.
